(19) United States
(12) Patent Application Publication
(10) Pub. No.: US 2024/0234115 A1
(43) Pub. Date: Jul. 11, 2024

(54) METHODS FOR RESOLVING CHARGE-STATE AMBIGUITIES IN HIGH AND ULTRA-HIGH MASS RANGE MASS SPECTRA

(71) Applicant: THERMO FINNIGAN LLC, San Jose, CA (US)

(72) Inventor: Paul R. Gazis, Mountain View, CA (US)

(21) Appl. No.: 18/151,321

(22) Filed: Jan. 6, 2023

(51) Int. Cl. H01J 49/00; G01N 33/68; H01J 49/02; H01J 49/16

(52) U.S. Cl. CPC H01J 49/0036; G01N 33/6848; H01J 49/025; H01J 49/165

(57) ABSTRACT

A mass spectrometry method comprises: identifying groups of charge state distributions (CSDs) within deconvoluted mass spectrometric data, wherein CSDs of each group comprise at least one common mass spectral peak that is assigned a different respective charge state within each CSD of each group; assigning, within each group, a respective weighting factor to each CSD; calculating, within each group and using the weighting factors, a score-weighted average molecular weight for each compound CSD; locating, within each group, a single target CSD that that corresponds to a molecular weight that is closest to the calculated average; for each common peak of each identified group, summing the intensities of the respective common assigned mass spectral peak across the group and assigning the summed intensity to the single target CSD of the group; discarding all CSDs other than the target CSDs; and calculating an abundance of each component compound using the summed intensities.

### TECHNICAL FIELD

The present invention relates to mass spectrometry and mass spectrometers. More particularly, the present invention relates to mass spectrometry of macromolecule compounds having molecular weights equal to or in excess of 450 kilodalton (450 kDa).

### BACKGROUND

Mass spectrometry has advanced over the last few decades to the point where it has become one of the most broadly applicable analytical tools for detection and characterization of a wide class of molecules. Mass spectrometric analysis is applicable to almost any molecule species that may be ionized so as to form an ion in the gas phase. Mass spectrometric analysis thus provides perhaps the most universally applicable method of quantitative analysis. In addition, mass spectrometry is a highly selective technique that is especially well-suited for the analysis of complex mixtures of different compounds in varying concentrations. Mass spectrometric methods provide very high detection sensitivities, approaching lower limits of detection of tenths of parts per trillion for some molecule species. As a result of these beneficial attributes, a great deal of attention has been directed over the last several decades at developing mass spectrometric methods for analyzing complex mixtures of biomolecules, such as peptides, proteins, carbohydrates, oligonucleotides as well as complexes of these molecules.

One common type of application of mass spectrometry to analyses of natural samples involves the characterization and/or quantification of components of complex mixtures of biomolecules. Many such biological molecules of interest are biopolymers, such as the polynucleotides (RNA and DNA), polypeptides and polysaccharides. Generally, the chemical composition (related to the specific collection of monomers of which the polymer is comprised) and the sequence of monomers are the distinguishing analytical characteristics of biopolymer molecules of a given class. Nonetheless, since biopolymer molecules of a given class generally have high molecular weights and can generate ions having a wide range of charge states, the process of distinguishing various molecules within a mixture of such molecules by mass spectrometry can be challenging.

Biomolecules are often introduced into an ionization source of a mass spectrometer dissolved within a mixture of water or aqueous buffers and organic solvents. Soluble sample-derived compounds may be separated from insoluble compounds using, for example, a solid-phase extraction device. The soluble fractions generally comprise a plurality of compounds, many of which are macromolecules, such as peptides and proteins. These fractions may then be further fractionated using reverse-phase chromatography. The various soluble biomolecules, either chromatographically separated or simply infused, may then be conveniently ionized by electrospray ionization. The ion species so generated are herein referred to as “primary” ion species. A mass analyzer of the mass spectrometer then detects and quantifies either the primary ion species or deliberately generated fragment ion species (or other product ion species) in accordance with the species' respective mass-to-charge (m/z) ratios.

An important feature of electrospray ionization is that it tends to preserve molecular structure without excessive fragmentation, as the ionization mechanism proceeds by adduction of solvent-derived charged units to each molecular framework. Thus, each organic molecule species of interest, [symbol] , in the un-ionized sample may give rise, after ionization, to a multitude of ion species, [symbol] , each such i^{th} ion species comprising a respective charge state, zᵢ. As a result, a mass spectrum is generally a highly complex record of a plurality of ion species generated from each one of a plurality of compounds.

More specifically, isotopically unresolved mass spectra of electrospray-generated ions of an organic molecule species, [symbol] , will generally exhibit a group of peaks, { [symbol] }, i=1,2,3, . . . , [symbol] , at various different mass-to-charge (m/z) values, where the peaks of a group are all associated with a same value of molecular weight, [symbol] , but are spread across a range of m/z values as a result of the fact that the various peaks correspond to ion species having a plurality of different charge states, zᵢ. Here, the notation [symbol] refers to the mass-to-charge value of the ion species group member that carries a total charge value, z, that is equal to i, this charge value also denoted as zᵢ. Also, the notation [symbol] refers to the greatest observed charge state in a mass spectrum of the organic molecule species, [symbol] . Under the assumption that the charge-carrying adduct species are primarily singly-charged (e.g., protons), then the P values of the [symbol] members of such a group of peaks are related by the expression

$${P}_{i}^{𝒦} = \frac{{M}^{𝒦} + {z}_{i} {m}_{A}}{{z}_{i}} = \frac{M}{{z}_{i}} + {m}_{A} \qquad Eq . ( 1 )$$

in which m_{A} is the mass of the adduct species. Each such group of peaks, { [symbol] }, is herein referred to as a “charge-state distribution”. The ability to recognize mass spectral peaks that correspond to multiply-charged ion species is useful when using mass spectrometry to recognize intact molecular ions of compounds having large molecular weights, such as molecular weights that are greater than approximately 450 kDa and that may be as great as several megadaltons. For such macromolecules, the m/z ratios of low-charge-state ions will generally be greater than the greatest m/z values that may be measured by many mass spectrometer systems. Increasing charge state, z, causes the mass spectral peaks to be observed at m/z values that are within the instrumental mass analysis range.

In practice, mass analysis of a single sample may give rise to many overlapping charge state distributions of the type noted above. Accordingly, many computer software programs and algorithms that are able to separate (“deconvolute”) and identify the various overlapping distributions are known and/or are commercially available. Generally, the output of such a deconvolution program comprises: (i) a listing of the centroid values of recognized peaks; (ii) a grouping of the centroid values into likely charge state distributions, with a likely charge state assigned for each peak of each group; and (iii), for each identified charge state distribution, a calculated molecular weight, [symbol] , of a molecular species, [symbol] , that corresponds to the respective charge state distribution.

For example, the methods employed by one such computer program are described in U.S. Pat. No. 10,217,619, the disclosure of which is hereby incorporated by reference in its entirety. FIG. 1 shows the deconvolution result from a five component protein mixture consisting of cytochrome c, lysozyme, myoglobin, trypsin inhibitor, and carbonic anhydrase as calculated by the methods described in U.S. Pat. No. 10,217,619. A top display panel 103 of the display shows the acquired data from the mass spectrometry represented as centroids. A centrally located main display panel 101 illustrates each peak as a respective symbol. The horizontally disposed mass-to-charge (m/z) scale 107 for both the top panel 103 and the central panel 101 is shown below the central panel. Each horizontal line within the main panel 101 connect centroid symbols of peaks that are assigned to a single respective charge state distribution. The numerical values attached to diagonal dotted lines within the panel 101 are the assigned charge states. The panel 105 on the left-hand side of the display shows the calculated molecular weight(s), in daltons, of the protein molecules. The molecular weight (MW) scale of the side panel 105 is oriented vertically on the display, which is perpendicular to the horizontally oriented m/z scale 107 that pertains to detected ions.

The above-described conventional mass analysis approach works well for proteins having low-to-moderate molecular weights. However, the present inventor has recognized the existence of a heretofore unrecognized problem that may arise as continued improvements in mass spectrometer performance extend the mass analysis range to greater m/z values. Specifically, as molecular weights approach and exceed approximately 450 kDa, ambiguities can arise in charge state determinations that can lead to false positive compound identifications as well as to incorrect determinations of abundances of compounds that are actually present in a sample. This ambiguity is due to the uncertainty, σ_{z}, in assigned charge state (e.g., standard deviation of assigned charge state) which can be shown to be related to the uncertainty in P by

$${σ}_{z} = \frac{2 z {σ}_{P}}{P} \qquad Eq . ( 2 )$$

where σₚ is the uncertainty in the mass-to-charge ratio, P, of a peak. As z and σₚ increase, the charge-state uncertainty 6σ_{z} of peaks of high-molecular-weight macromolecules can approach or exceed 1, thereby leading to mis-assigned charge states. This uncertainty is a natural consequence of the fact that the m/z spacing between adjacent peaks of a charge state distribution decreases with increasing z. When this happens, some of the signal from a charge state distribution associated with molecular species, [symbol] , may be incorrectly assigned to a charge state distribution of a different, non-present or non-existent molecular species, [symbol] . As a consequence, the abundance of the true (actually present) species, [symbol] , will be under-reported, since a portion of its mass spectral signal will be incorrectly assigned to the falsely-identified species, [symbol] . Additionally, the false positive identification(s) may cause inaccuracies in subsequent actions or decisions—for example, medical diagnoses—that rely on the mass spectral data.

### SUMMARY

A method is described to resolve the ambiguity in charge state determination that occurs when deconvoluting ultra-high-mass (equal to or in excess of 450 kDa) mass spectrometry data. This method identifies component compounds identified by the deconvolution routine that share one or more assigned m/z peaks but within which the shared m/z peaks are assigned different charge states in the different component compounds. The method further determines which components are real and which are false positives. The method then discards the false positives and combines their signals with the signals of the actually-present components to correct the abundances of the various components and to, optionally, generate a final spectrum of molecular weights of the components.

According to an aspect of the present teachings, a method of eliminating false positive identifications and correcting abundances, as determined by a deconvolution of mass spectrometric data, of component compounds of a sample that have molecular weights greater than or equal to approximately 450 kDa is provided, the method comprising:
identifying a group of charge state distributions, as determined by the deconvolution, within the deconvoluted mass spectrometric data, wherein all charge state distributions of the identified group comprise at least one common assigned mass spectral peak and wherein each common assigned mass spectral peak is assigned a different respective charge state within each charge state distribution of the group;
recognizing charge state distributions within the identified group of charge state distributions that correspond to false-positive compound identifications;
summing peak intensities of common assigned mass spectral peaks of the identified group of charge state distributions that are identified to correspond to false-positive compound identifications together with peak intensities of a target charge state distribution of the group that is not identified as corresponding to a false-positive compound identification;
discarding, from the identified group of charge state distributions, all charge state distributions that correspond to the false-positive compound identifications; and
calculating an abundance of a component compound corresponding to the target charge state distribution using the summed peak intensities.

In some instances, the step of identifying charge state distributions within the recognized group of charge state distributions that correspond to false-positive compound identifications may comprise:
assigning a respective weighting factor to each charge state distribution of the identified group of charge state distributions;
calculating, within the identified group of charge state distributions, a score-weighted average molecular weight for each real or hypothetical component molecular species that corresponds to a respective charge state distribution of the identified group of charge state distributions, the calculating using the assigned weighting factors; and
locating, within the identified group of charge state distributions, the target charge state distribution as the charge state distribution that is closest in value to the calculated average molecular weight.
   In some instances, the weighting factors may be assigned based, at least in part, on assigned or calculated mean-square errors in calculated molecular weights of real or hypothetical component molecular species that correspond to the charge state distributions. In some instances, the weighting factor may be assigned based, at least in part, on the intensities of mass spectral peaks of the respective charge state distribution.

According to another aspect of the present teachings, a mass spectrometer system is provided, comprising:
an electrospray ion source configured to receive a sample comprising one or more component compounds having molecular weights that are greater than or equal to 450 kilodaltons (kDa);
a mass analyzer configured to receive ions generated by ionization of component compounds of the sample;
a detector configured to detect ions output from the mass analyzer and to generate mass spectral data therefrom;
a data storage device configured to receive the mass spectral data from the detector; and
a programmable processor device configured to receive the mass spectral data from either the detector or the data storage device and comprising computer readable instructions operable to:
   perform a conventional deconvolution of the mass spectral data; and
   automatically detect and eliminate false positive compound identifications generated by the conventional deconvolution, wherein the false compound identifications are caused by standard deviations of charge state assignments being equal to or greater than unity.

### BRIEF DESCRIPTION OF THE DRAWINGS

The above noted and various other aspects of the present invention will become apparent from the following description which is given by way of example only and with reference to the accompanying drawings, not necessarily drawn to scale, in which:

FIG. 1 is a graphical depiction of a mass spectral deconvolution result obtained from a five-component protein mixture consisting of cytochrome c, lysozyme, myoglobin, trypsin inhibitor, and carbonic anhydrase;

FIG. 2A is a chromatogram exhibiting the elution profile of various multimers of the globular protein apoferritin;

FIG. 2B is mass low-resolution mass spectrum of the eluate corresponding to the elution profile of FIG. 2A;

FIG. 3A is a spectrum of calculated molecular weights of eluting components, the molecular weights generated by application of a deconvolution algorithm to the data of FIG. 2B;

FIG. 3B is a graphical depiction of the manner by which charge state uncertainty may cause individual peaks of the mass spectral data of FIG. 2B to be assigned to more than one charge state distribution, the graphical depiction including centrally located main display panel that illustrates each peak assignment as a respective symbol, a top display panel showing the raw mass spectral data, a left-hand side panel showing calculated molecular weight(s) along a vertical molecular-weight axis, and a horizontally disposed mass-to-charge (m/z) scale for both the top panel and the central panel;

FIG. 3C is a graphical depiction, similar to FIG. 3B, that shows the results of correction of the mass spectral data to a single molecular weight value, using the methods of the present teachings;

FIG. 3D is a spectrum of corrected molecular weights of the eluting components of FIG. 3A, as corrected in accordance with the present teachings;

FIG. 4. is a flow diagram of a method for resolving mass spectrometric charge-state ambiguities in accordance with the present teachings; and

FIG. 5 is a schematic diagram of a system for generating and automatically analyzing chromatography/mass spectrometry spectra as may be employed in conjunction with methods of the present teachings.

### DETAILED DESCRIPTION

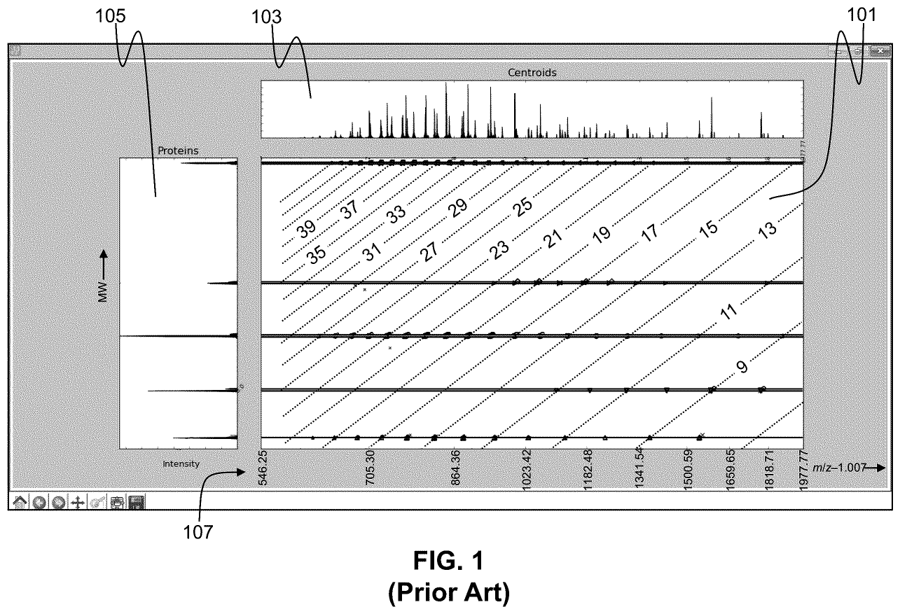

The following description is presented to enable any person skilled in the art to make and use the invention, and is provided in the context of a particular application and its requirements. Various modifications to the described embodiments will be readily apparent to those skilled in the art and the generic principles herein may be applied to other embodiments. Thus, the present invention is not intended to be limited to the embodiments and examples shown but is to be accorded the widest possible scope in accordance with the features and principles shown and described. To fully appreciate the features of the present invention in greater detail, please refer to FIGS. 1, 2A, 2B, 3A, 3B, 3C, 3D, 4 and 5 in conjunction with the following description.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that, for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements.

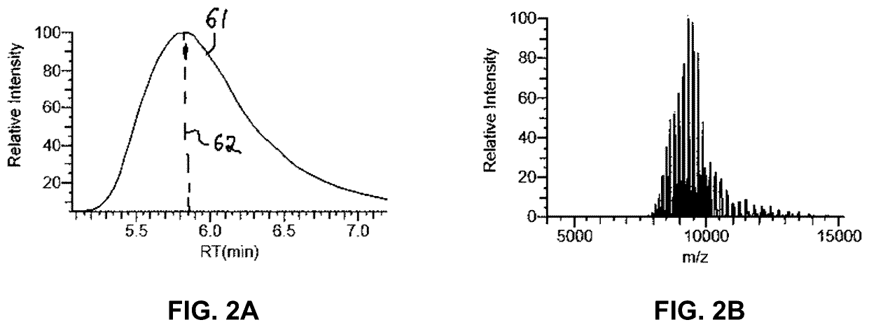

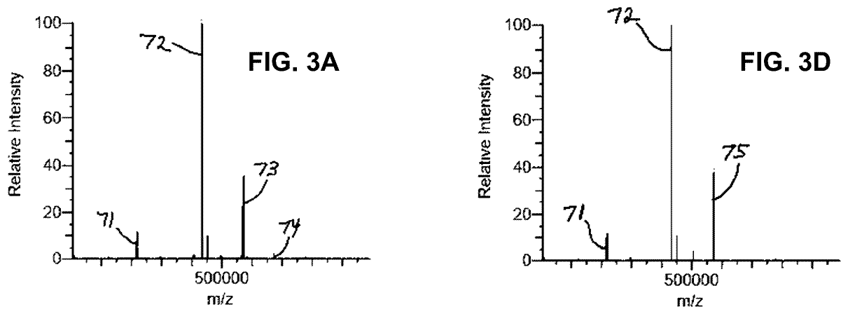

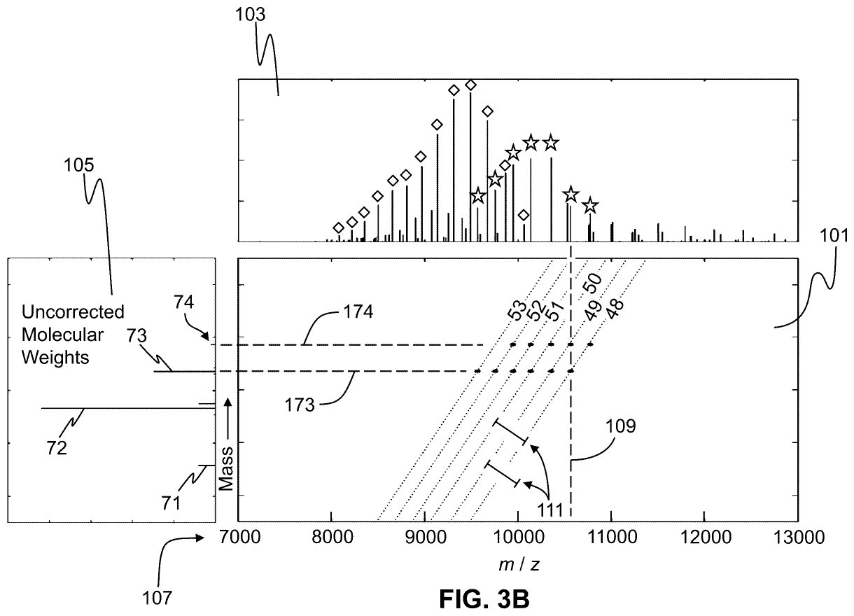

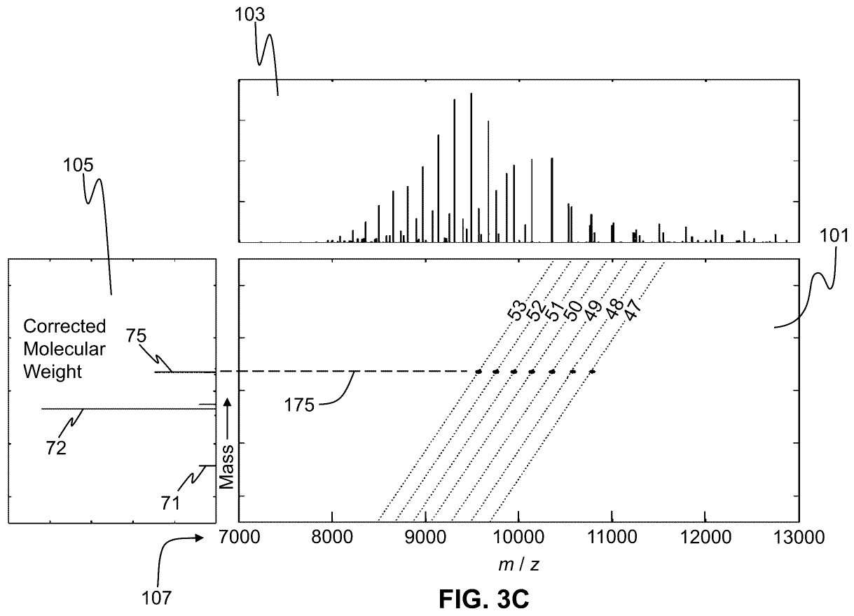

FIGS. 2A-2B and 3A-3D illustrate an example of the occurrence of the heretofore unrecognized problem discussed above. FIG. 2A is a chromatogram exhibiting the elution profile 61 of various multimers of the globular protein apoferritin and comprising a broad elution peak that is maximized at retention time 62. FIG. 2B is low-resolution mass spectrum, within the m/z range 4000-15000 Th, of the eluate corresponding to the elution profile of FIG. 2A. FIG. 3A is a spectrum of the molecular weights of eluting chemical components of the eluate, as determined by application of a conventional deconvolution algorithm to the data of FIG. 2B. The deconvolution routine calculates chemical components having molecular weights of 471679.1 Da (molecular component peak 71), 493192.1 Da (molecular component peak 72) 507043.7 Da (molecular component peak 73) and 517193.1 Da (molecular component peak 74). The calculated molecular weight of molecular component peak 73 is close to the known molecular weight of the apoferritin 24-mer. However, as is evident from the charge-state assignments listed in Table 1 below, some of the mass spectrometric signal intensity from the apoferritin 24-mer peaks (i.e., the data depicted in FIG. 2B) has been assigned, in this example, to a false positive, represented as molecular component peak 74. This conclusion is indicated by the fact that the deconvolution routine, in this instance, assigns each of several observed mass spectrometric peaks (listed as mass spectrometric peaks 2-5 in the table) to both the true molecular component peak 73 as well as to the false molecular component peak 74 as a result of assigning two different charge states to each such mass spectrometric peak. Notably, each of the charge state assignments that are used in the false identification of molecular component peak 74 are consistently 1 unit greater than the assignments for the true molecular component peak 73. The ambiguities in the charge state assignments result from the loss of precision in the charge state assignment, as noted in Eq. (2).

TABLE 1
Charge State Assignments

| | | z assignment for | z assignment for |
|---|---|---|---|
| | m/z | component 73 | component 74 |
| 1 | 10,772.97 | — | 48 |
| 2 | 10,563.92 | 48 | 49 |
| 3 | 10,353.14 | 49 | 50 |
| 4 | 10,136.58 | 50 | 51 |
| 5 | 9,945.68 | 51 | 52 |
| 6 | 9,752.95 | 52 | — |
| 7 | 9,565.44 | 53 | — |

FIG. 3B is a graphical depiction, similar to the graphical depiction of FIG. 1, that shows how charge state uncertainty may cause individual peaks of the mass spectral data of FIG. 2B to be assigned to more than one charge state distribution. The top display panel 103 of FIG. 3B is an expanded version of the mass spectral data of FIG. 2B, referenced to the horizontal mass-to-charge (m/z) scale 107 at the bottom of the diagram. The left-hand side panel 105 shows a rotated view of the calculated molecular weights, as calculated by a standard deconvolution software package, with the mass scale (i.e., the molecular-weight scale) oriented vertically. Finally, the center panel 101 relates the mass spectral peaks of Table 1 to the assigned charge states as well as to the horizontally disposed m/z scale and the vertically disposed molecular weight scale, with the centroids of the peaks of Table 1 illustrated as individual dots within center panel 101. In similarity to the graphical depiction of FIG. 1, the diagonal dotted lines in center panel 101 are lines of constant charge state, with each such diagonal line labeled in accordance with the respective charge state, zᵢ, that it represents. Each diagonal dotted line has a respective slope of 1/zᵢ.

Stars located above the mass spectrum in top panel 103 of FIG. 3B indicate the locations of the seven different peaks used in the assignments, as tabulated in Table 1. (For reference, the peaks marked with diamonds in the top panel 103 of FIG. 3B represent a charge state distribution of another multimer of apoferritin having a molecular weight of 493 kDa, represented by molecular component peak 72 in FIGS. 3A-3D). Although only seven peaks are tabulated in Table 1, these seven peaks are plotted using 11 different points within the molecular weight versus mass-to-charge plot of the central panel 101 of FIG. 3B because four of the peaks (peaks 2-5 of Table 1) are assigned, by the deconvolution, to two hypothetical peaks 73, 74 that correspond to different molecular weights, as noted above. Horizontal line 173 connects the peak 73 to the corresponding charge state assignments of the tabulated mass spectral lines and horizontal line 174 connects the peak 74 to its corresponding charge state assignments, both sets of assignments as calculated by the deconvolution.

As noted above (e.g., Eq. 2), the uncertainty in charge assignments can approach and exceed one unit of charge when ions of compounds having molecular weights approaching or exceeding 500 kDa are being investigated by mass spectrometry. Charge assignment error bars may be defined in various ways, e.g., in terms of Op as defined in Eq. 2 or multiples thereof. The exact molecular weight threshold at which the charge uncertainty exceeds one charge unit will depend upon how the error bars are defined as well as upon various instrumental factors, such as the reproducibility and/or precision of mass spectrometric m/z measurements, m/z detection range, the magnitude of charge states being detected (e.g., ≥50, ≥100, ≥150), etc. Hypothetical charge-state error bars 111 depicted in FIG. 3B illustrate a range within which the diagonal dotted iso-charge-state lines may vary, with statistical plausibility, when the charge-state uncertainty (however defined) is +1 charge unit. Accordingly, without making use of additional information, either one or both of the sets of charge state assignments—e.g., either aligned with line 174 or aligned with line 173—must be considered to be statistically plausible.

FIG. 3C is a graphical depiction, similar to FIG. 3B, that shows the results of correction of the mass spectral data to a single molecular weight value, using the methods of the present teachings, as further described below. Using these methods, the molecular component peak 74 is identified as a false positive and the adjusted molecular weight of the corrected actual peak 75 is 507,353.41 Da (as compared to 507043.7 Da originally calculated for peak 73 and 517193.1 Da originally calculated for false-positive peak 74).

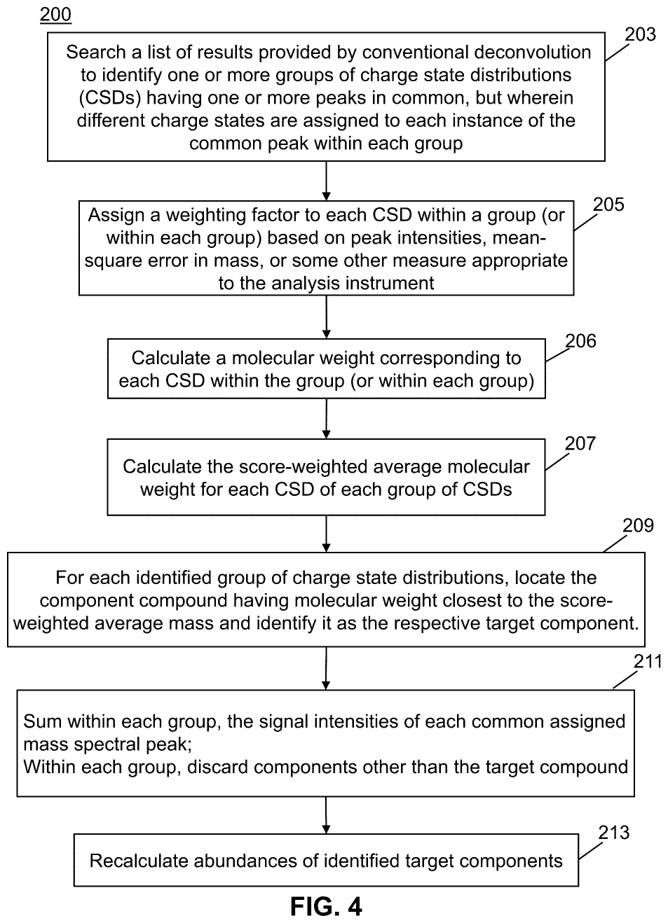

FIG. 4 is a flow diagram of a method 200 for resolving such mass spectrometric charge-state ambiguities in accordance with the present teachings. The method 200 is applicable to analyses of macromolecules having molecular weights greater than or equal to 450 kDa, that are ionized by an ionization technique in which ions of the macromolecules are generated by adduction of multiple charged particles to the analyte molecules. Generally, but not necessarily, the method pertains to analysis of organic macromolecules that are ionized by either electrospray or thermospray ionization.

In preparation for execution of the method 200, a mass spectrum of a sample containing macromolecular component compounds is measured by a mass spectrometer and/or retrieved from data storage prior to the execution of the method 100. Also, after measurement or and/retrieval of the mass spectrum, a conventional “deconvolution” procedure in which the various mass-to-charge ratios, P, of the observed mass spectral peaks are logically organized into groups of peaks, { [symbol] }, { [symbol] }, { [symbol] }, . . . that are tentatively considered to comprise charge state distributions that correspond, respectively, to tentatively identified component molecule species, [symbol] , [symbol] , [symbol] , etc. As noted above, charge states are assigned to each member of each charge state distribution and molecular weights are assigned to each component molecule species as part of the deconvolution procedure. In the practice of High Mass Range mass spectrometry and Ultra High Mass range mass spectrometry, the molecular weights of the various component molecule species may range from 0.45 MDa (megadaltons) up to 3 MDa whereas the m/z values of the detected ion species may be in the range of 4000-20000 Th. Accordingly, the charge states of the detected ions may range from 50 up to 150. Accordingly, the assignments of charge states by the deconvolution routine must be considered as tentative due to the increase in charge assignment uncertainty noted above. As a result, some of the identified component molecule species may be false-positive identifications and some determined abundances of actually present species may be underestimated.

In the initial step 203 of the method 200, the listing of results generated in by the deconvolution procedure is searched to identify all instances in which one or more observed mass spectral peak(s) is/are assigned, by the deconvolution routine, to a plurality of charge-state distributions (i.e., a “group” of charge state distributions) and is/are assigned different respective charge states within each charge state distribution of the group. Based on this search, one or more groups of charge state distributions within the deconvoluted mass spectrometric data are identified, wherein the criteria for identifying a group are that all charge state distributions of the group comprise at least one common assigned mass spectral peak (i.e., a peak that is shared by all charge state distributions of the group) and, further, that each common assigned mass spectral peak is assigned a different respective charge state within each charge state distribution.

In step 205, a weighting factor is assigned to each charge state distribution of each identified group of charge state distributions (step 203), each weighting factor relating to the quality of the mass spectral data of the mass spectral peaks that correspond to the species, according to some chosen metric. The weighting factor may be based on either mass spectral peak intensity, mean-square error in mass, or some other quality metric that is appropriate to the analysis instrument.

In step 207, the weighting factors are employed to calculate a score-weighted average molecular weight for each group of charge state distributions that are identified in step 203. However, prior to the execution of the step 207, the step 206 comprises first calculating, using all of the P and z values of mass spectral peaks within each charge state distribution of each identified group, a molecular weight for a component compound that corresponds to that charge state distribution, regardless of whether or not the component compound is an actual compound or a hypothetical compound and regardless of whether or not the component compound is actually present in the sample. These individual molecular weights are then averaged in step 207, using the weighting factors assigned in step 205.

Although each score-weighted average molecular weight that is calculated in step 207 generally does not correspond to any real component species, it will generally be close to the true molecular weight of a specific species that is indeed present in the sample. Thus, in step 209, the component molecular species for which the originally calculated molecular weight (step 206) is closest to the intensity-weighted average molecular weight (step 207) of each group of charge state distributions is located from among the group. This so-located individual molecular component species within each group is the most-likely true positive species of the group and is herein referred to as the “target” component species.

In subsequent step 211, all component species other than the identified target component are discarded from the from each identified group. The discarded component molecular species are considered to be false positives. Accordingly, for each common assigned peak of each group of charge state distributions, the signal intensity that was originally assigned to the discarded species is summed to the signal intensity of the target component species, as located in the previous step. Finally, in step 213, the abundances of various identified target components are recalculated from the respective summed peak signal intensities.

FIG. 3B shows the results when this method is applied to the deconvolution results shown in FIG. 3A. Specifically, the false positive at 517 kDa (peak 74 in FIG. 3A) is identified, discarded, and its signal combined with the true component at 507 kDa to yield a revised mass and intensity, depicted in FIG. 3B as molecular component peak 75.

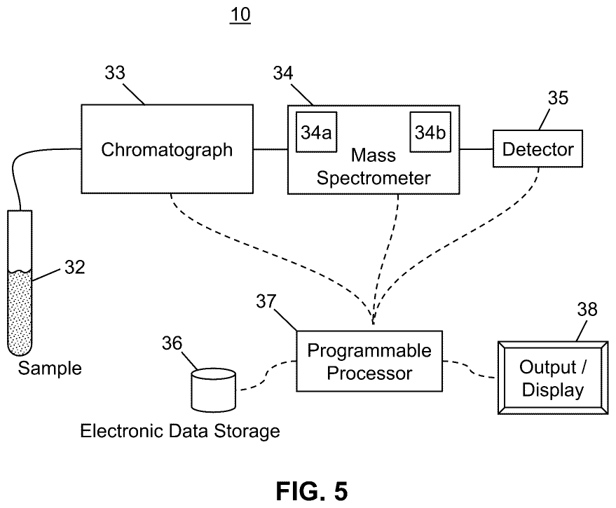

FIG. 5 is a schematic diagram of a system 10 for generating and automatically analyzing chromatography/mass spectrometry spectra in accordance with the present teachings. A chromatograph 33, such as a liquid chromatograph, high-performance liquid chromatograph or ultra-high performance liquid chromatograph receives a sample 32 of an analyte mixture and at least partially separates the analyte mixture into individual chemical components, in accordance with well-known chromatographic principles. The at least partially separated chemical components are transferred to a mass spectrometer system 34 at different respective times for mass analysis. As each chemical component is received by the mass spectrometer, it is ionized by an ionization source 34a of the mass spectrometer system. The ionization source may produce a plurality of ion species (i.e., a plurality of precursor ion species) comprising differing charges or masses from each chemical component. Thus, a plurality of ion species of differing mass-to-charge ratios (e.g., a charge state distribution) may be produced for each chemical component, each such component eluting from the chromatograph at its own characteristic time. These various ion species are analyzed by a mass analyzer 34b of the mass spectrometer system 34 and detected an ion detector 35. As is well known, through the combined effects of mass analysis and ion detection, the mass spectrometer generates mass spectral data, which provides a record of detected ion intensity as a function of mass-to-charge ratio of ions generated from a sample. Using the mass spectral data, various ion species may be appropriately identified according to their various mass-to-charge ratios. The mass spectrometer may comprise a mass filtering apparatus (not shown) that isolates ion species within certain selected m/z ranges as well as a fragmentation cell (not shown) for generation of product ions by fragmentation of the selected ion species.

Still referring to FIG. 5, a programmable processor 37 of the system 10 is electronically coupled to the detector of the mass spectrometer and receives the data produced by the detector during chromatographic/mass spectrometric analysis of the sample(s). The programmable processor may comprise a separate stand-alone computer or may simply comprise a circuit board or any other programmable logic device operated by either firmware or software. Optionally, the programmable processor may also be electronically coupled to the chromatograph and/or the mass spectrometer in order to transmit electronic control signals to one or the other of these instruments so as to control their operation. The nature of such control signals may possibly be determined in response to the data transmitted from the detector to the programmable processor or to the analysis of that data. The programmable processor may also be electronically coupled to a display or other output 38, for direct output of data or data analysis results to a user, or to electronic data storage 36.

The programmable processor 37 of the system 10 shown in FIG. 5 generally comprises computer-readable instructions that are operable to: control individual operations and sequences of operations of the chromatograph 33; control operations and sequences of operations of the mass spectrometer 34; receive a mass spectrum from the detector 35; perform a conventional deconvolution procedure of the data of the mass spectrum; and perform the logical steps of the method 100 (FIG. 4) in order to eliminate false positive identifications and correct compound abundances provided by the conventional deconvolution procedure.

The discussion included in this application is intended to serve as a basic description. The present invention is not intended to be limited in scope by the specific embodiments described herein, which are intended as single illustrations of individual aspects of the invention. Functionally equivalent methods and components are within the scope of the invention. Various other modifications of the invention, in addition to those shown and described herein will become apparent to those skilled in the art from the foregoing description and accompanying drawings.

What is claimed is:

1. A method of eliminating false positive identifications and correcting an abundance, as determined from deconvoluted mass spectrometric data, of a component compound of a sample having a molecular weight greater than or equal to 450 kDa, the method comprising:
(a) identifying a group of charge state distributions recognized by the deconvolution within the deconvoluted mass spectrometric data, wherein all charge state distributions of the identified group comprise at least one common assigned mass spectral peak and wherein each common assigned mass spectral peak is assigned a different respective charge state within each charge state distribution of the group;
(b) recognizing charge state distributions within the identified group of charge state distributions that correspond to false-positive compound identifications;
(c) summing peak intensities of common assigned mass spectral peaks of the identified group of charge state distributions that are identified to correspond to false-positive compound identifications together with peak intensities of a target charge state distribution of the group that is not identified as corresponding to a false-positive compound identification;
(d) discarding, from the identified group of charge state distributions, all charge state distributions that correspond to the false-positive compound identifications; and
(e) calculating an abundance of a component compound corresponding to the target charge state distribution using the summed peak intensities.

2. A method as recited in claim 1, wherein the step (b) of recognizing charge state distributions within the identified group of charge state distributions that correspond to false-positive compound identifications comprises:
(b1) assigning a respective weighting factor to each charge state distribution of the identified group of charge state distributions;
(b2) calculating, within the identified group of charge state distributions, a score-weighted average molecular weight for each real or hypothetical component molecular species that corresponds to a respective charge state distribution of the identified group of charge state distributions, the calculating using the assigned weighting factors; and
(b3) locating, within the identified group of charge state distributions, the target charge state distribution as the charge state distribution that is closest in value to the calculated average molecular weight.

3. A method as recited in claim 2, wherein each weighting factor is assigned based, at least in part, on the intensities of mass spectral peaks of the respective charge state distribution.

4. A method as recited in claim 2, wherein each weighting factor is assigned based, at least in part, on an assigned or calculated mean-square error in the molecular weight of the real or hypothetical component molecular species that corresponds to the respective charge state distribution.

5. A mass spectrometer system comprising:
an electrospray ion source configured to receive a sample comprising one or more component compounds having molecular weights that are greater than or equal to 450 kiloDaltons (kDa);
a mass analyzer configured to receive ions generated by ionization of component compounds of the sample;
a detector configured to detect ions output from the mass analyzer and to generate mass spectral data therefrom;
a data storage device configured to receive the mass spectral data from the detector; and
a programmable processor device configured to receive the mass spectral data from either the detector or the data storage device and comprising computer readable instructions operable to:
perform a conventional deconvolution of the mass spectral data; and
automatically detect and eliminate false positive compound identifications generated by the conventional deconvolution, wherein the false compound identifications are caused by standard deviations of charge state assignments generated by the conventional deconvolution being equal to or greater than unity.

6. A mass spectrometer system as recited in claim 5, wherein computer readable instructions that are operable to automatically detect and eliminate false positive compound identifications generated by the conventional deconvolution are operable to:
identify a group of charge state distributions, generated by the deconvolution, within the deconvoluted mass spectrometric data, wherein all charge state distributions of the identified group comprise at least one common assigned mass spectral peak and wherein each common assigned mass spectral peak is assigned a different respective charge state within each charge state distribution of the group;
assign, within the identified group of charge state distributions, a respective weighting factor to each charge state distribution of the group;
calculate, within the identified group of charge state distributions and using the weighting factors, a score-weighted average molecular weight for each real or hypothetical component molecular species that corresponds to a respective identified charge state distribution of the group;
locate, within the identified group of charge state distributions, a single target charge state distribution that that corresponds to a molecular weight the that is closest in value to the calculated average molecular weight; and
eliminate, from the identified group of charge state distributions, all charge state distributions other than the single target charge state distribution.

7. A mass spectrometer system as recited in claim 5, wherein the charge states of the detected ions of the one or more component compounds are greater than or equal to 50.

8. A mass spectrometer system as recited in claim 5, wherein the charge states of the detected ions of the one or more component compounds are greater than or equal to 100.
